(19) United States
(12) Patent Application Publication
(10) Pub. No.: US 2019/0054397 A1
(43) Pub. Date: Feb. 21, 2019

(54) PROTEIN PURIFICATION DEVICE

(71) Applicant: Sartorius Stedim Biotech GmbH, Goettingen (DE)

(72) Inventors: Karl Pflanz, Goettingen (DE); Andreas Pickl, Klein Schneen (DE); Holger Linne, Goettingen (DE); Vanessa Ransiek, Goettingen (DE); Kathrin Schroeder-Tittmann, Goettingen (DE)

(21) Appl. No.: 16/102,989

(22) Filed: Aug. 14, 2018

(30) Foreign Application Priority Data
Aug. 17, 2017 (DE) 102017007808.4

(51) Int. Cl. B01D 15/34; B01D 15/12; B01D 15/18

(52) U.S. Cl. CPC B01D 15/34; B01J 20/28004; B01D 15/1871; B01D 15/125

(57) ABSTRACT

A device for purifying protein from a protein-containing fluid includes a prefilter unit, which has at least one size exclusion filter, a membrane adsorber unit, which has at least one porous membrane with functional constituents which adsorb the protein to be purified, and a collecting container. The collecting container, the membrane adsorber unit and the prefilter unit are configured such that the collecting container is suitable for receiving the membrane adsorber unit; and the membrane adsorber unit is suitable for receiving the prefilter unit. An associated method for purifying protein from a protein-containing fluid using the device is also disclosed.

### CROSS REFERENCE TO RELATED APPLICATIONS

The following disclosure is based on and claims the benefit of and priority under 35 U.S.C. § 119(a) to German Patent Application No. DE 102017007808.4, filed Aug. 17, 2017, which is incorporated in its entirety into the present application by reference.

### FIELD OF THE INVENTION

An aspect of the present invention relates to a device for purifying protein from a protein-containing fluid as well as to a method for purifying protein from a protein-containing fluid and to an application of the device for purifying protein from a protein-containing fluid.

### BACKGROUND

In biotechnology, living cells are often used to produce large amounts of protein. For this purpose, the genes, which serve as the building blocks for the desired proteins, are cloned into the appropriate plasmids and brought into the cells for expression. The protein, which is produced intracellularly in this way, can be isolated from the cell plasma by purification after cell lysis. If the protein is secreted by the cell into the surrounding medium, there is no need for cell lysis.

The prior art discloses isolation/purification methods, which typically follow a standard routine: A suspension of cells that contains the desired protein is centrifuged; and the supernatant is discarded. The resulting cell pellet is resuspended in the buffer; and the resuspended cells are mechanically or chemically lysed. Then after a second centrifugation step to remove the cell debris, the supernatant is separated by a column, filled with (gelatinous) chromatographic material, in particular, using chromatographic materials that selectively bind the desired protein. After completion of the chromatography process with a subsequent washing step, the bound protein can be detached from the column with the elution buffer solution and collected.

However, the foregoing method is associated with a number of drawbacks. In order to achieve reasonable flow rates in column chromatography, said column chromatography is often carried out in a vacuum or under negative pressure. However, this method increases the amount of equipment that is needed, since a connecting system for generating the negative pressure has to be present. In addition, negative pressure has an adverse effect on the protein solution to be purified, because this protein solution often foams under negative pressure; and the protein to be clarified settles out.

For larger amounts of protein to be purified, a standard FPLC system (“fast protein liquid chromatography”; chromatography under pressure ranging from typically 0.2 to 10 bar) is usually used. However, a plethora of individual steps, such as a preceding centrifugation process, loading onto a column, etc., are necessary.

Furthermore, gelatinous chromatographic material has to be stored immersed in a liquid and under cool conditions, in order to maintain its stability. However, this requirement leads to a higher risk of bacterial contamination, especially since such chromatographic material cannot be sterilized by simple methods.

In order to avoid the use of negative pressure, chromatographic methods have been proposed, in which the flow rate is increased through centrifugation. However, the so-called spin columns are suitable only for purifying samples having a small amount of fluid, on account of the length of the column required for effective chromatographic separation. For example, the maximum volume that commercially available spin columns can receive and purify ranges from about 0.5 to about 2 ml.

Based on the drawbacks described above, an object of the present invention is to provide a simple device that makes it possible to purify protein from a protein-containing fluid quickly (i.e., with few process steps), effectively (i.e., a high yield of purified protein per cell unit), reliably and aseptically as well as to provide a method, which uses this device, and an application of the device.

### SUMMARY

This object is achieved by embodiments of the present invention that are characterized in the claims.

In particular, according to one aspect of the invention, a device is provided for purifying protein from a protein-containing fluid, said device comprising

a cylindrically shaped prefilter unit (1), which has at least one size exclusion filter (4);

a cylindrically shaped membrane adsorber unit (2), which has at least one porous membrane (5) with functional constituents, which adsorb the protein to be purified; and

a cylindrically shaped collecting container (3);
wherein the collecting container (3), the membrane adsorber unit (2) and the prefilter unit (1) are configured in such a way that the collecting container (3) is suitable for receiving the membrane adsorber unit (2); and the membrane adsorber unit (2) is suitable for receiving the prefilter unit (1).

The term “protein-containing fluid” is defined here as any fluid that contains protein. In particular, the devices and methods are suitable for purifying a protein-containing cell broth, i.e., a solution or suspension, which comprises the cells and/or fragments of chemically or mechanically lysed cells, which have produced the protein to be purified, and the protein to be purified.

The protein to be purified is not subject to any particular constraint. For example, proteins, such as antibodies or tagged (marked) proteins, for example, His-tagged (His-marked) proteins, can be purified with the device provided herein.

An additional aspect of the present invention relates to a method for purifying protein from a protein-containing fluid, said method comprising the steps of:
(a) providing a device for purifying protein from a protein-containing fluid, wherein a prefilter unit (1) is located inside or partially inside a membrane adsorber unit (2), and the membrane adsorber unit (2) is located inside or partially inside a collecting container (3);
(b) filling the prefilter unit (1) with the protein-containing fluid;
(c) centrifuging the device, wherein constituents of the protein-containing fluid that cake a porous membrane (5) of the membrane adsorber unit (2) are retained by at least one size exclusion filter (4) of the prefilter unit (1); the protein to be purified is adsorbed on the porous membrane (5) of the membrane adsorber unit (2); and a filtrate collects in the collecting container (3);
(d) discarding the filtrate located in the collecting container (3);
(f) filling the membrane adsorber unit (2) with elution buffer solution;
(g) eluting the protein, adsorbed on the porous membrane (5), with the elution buffer solution in the collecting container (3) to obtain the purified protein.

### BRIEF DESCRIPTION OF THE DRAWINGS

The device, which is configured for purifying protein by centrifugation, is described in greater detail below with reference to a preferred embodiment, in which:

FIG. 1 shows the components of a device provided herein as single parts.

FIGS. 2A-2B show devices provided herein in the “fitted-together state”. In FIG. 2A, in this fitted-together state, the prefilter unit is located completely inside the membrane adsorber unit, and the membrane adsorber unit is located partially inside the collecting container. In FIG. 2B, in this fitted-together state, the prefilter unit is located completely inside the membrane adsorber unit, and the membrane adsorber unit is located completely inside the collecting container.

FIG. 3 defines the terms “inner wall cylinder diameter”, “outer wall cylinder diameter” and “length” of the components of the device provided herein.

### DETAILED DESCRIPTION

The device provided herein comprises a cylindrically shaped prefilter unit (1), a cylindrically shaped membrane adsorber unit (2) and a cylindrically shaped collecting container (3). The term “cylindrically shaped” shall mean in the context of the present application that the prefilter unit (1), the membrane adsorber unit (2) and the collecting container (3) are hollow bodies that are defined by cylindrically shaped walls.

More particularly, the device for purifying protein from a protein-containing fluid comprises:

a cylindrically shaped prefilter unit (1), which has at least one size exclusion filter (4);

a cylindrically shaped membrane adsorber unit (2), which has at least one porous membrane (5) with functional constituents that adsorb the protein to be purified; and

a cylindrically shaped collecting container (3);

wherein the collecting container (3), the membrane adsorber unit (2) and the prefilter unit (1) are configured in such a way that the collecting container (3) is suitable for receiving the membrane adsorber unit (2); and the membrane adsorber unit (2) is suitable for receiving the prefilter unit (1).

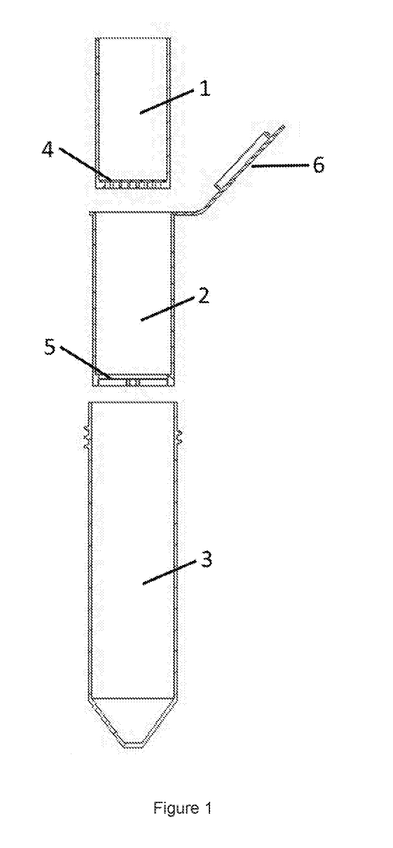

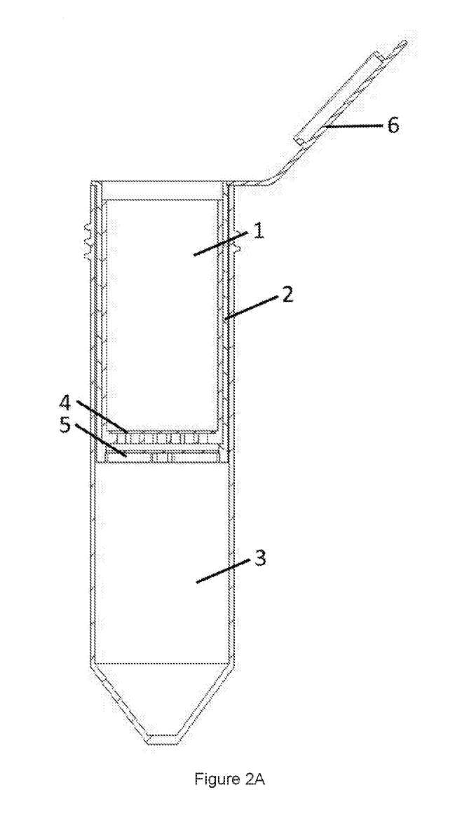

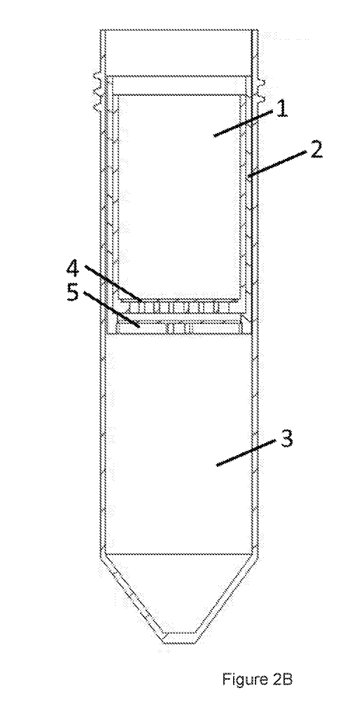

FIG. 1 shows the components of the device as single, disassembled parts. FIGS. 2A-2B show the device in the “fitted-together state”. According to FIG. 2A, in this fitted-together state, the prefilter unit (1) is located completely inside the membrane adsorber unit (2); and the membrane adsorber unit (2) in turn is located partially inside the collecting container (3). According to FIG. 2B, in this fitted-together state, the prefilter unit (1) is located completely inside the membrane adsorber unit (2); and the membrane adsorber unit (2) in turn is located completely inside the collecting container (3). In this fitted-together state the device is configured for centrifugation in order to purify protein from a protein-containing fluid, wherein the protein may be found in the prefilter unit (1) prior to centrifugation.

The material of the cylindrically shaped walls is not subject to any particular constraint; and it is possible to use, for example, any customary material for the centrifugation containers. According to a preferred embodiment, the material of the cylindrically shaped walls comprises endotoxin-free and gamma-sterilizable plastics, such as, for example, polypropylene, polycarbonate, styrene-containing plastics, such as acrylonitrile butadiene styrene(ABS), polyether ether ketone(PEEK), polyamide, polyethylene, polybutylene and polyethylene terephthalate(PET).

The wall thickness of the prefilter unit (1), the membrane adsorber unit (2) and collecting container (3) is not subject to any particular constraint. According to a preferred embodiment , the respective wall thicknesses are in a range of 0.25 mm to 5 mm, more preferably 0.6 mm to 2 mm.

The collecting container (3), the membrane adsorber unit (2) and the prefilter unit (1) are configured in such a way that the collecting container (3) is suitable for receiving the membrane adsorber unit (2); and the membrane adsorber unit (2) is suitable for receiving the prefilter unit (1). The expression “suitable for receiving” shall mean in the context of the present application that one component can be inserted either completely or partially into another component, i.e., can be located either completely or partially in the cylindrical interior of the other component. According to a particularly preferred embodiment, the collecting container (3) is suitable for receiving the membrane adsorber unit (2) in its entirety; and the membrane adsorber unit (2) is suitable for receiving the prefilter unit (1) in its entirety, such that the device provided herein can be closed with an end cap (6). Optionally the device provided herein also comprises a clamping ring, which securely clamps the porous membrane (5) in the membrane adsorber unit (2) and serves at the same time as a spacer between the membrane adsorber unit (2) and the prefilter unit (1).

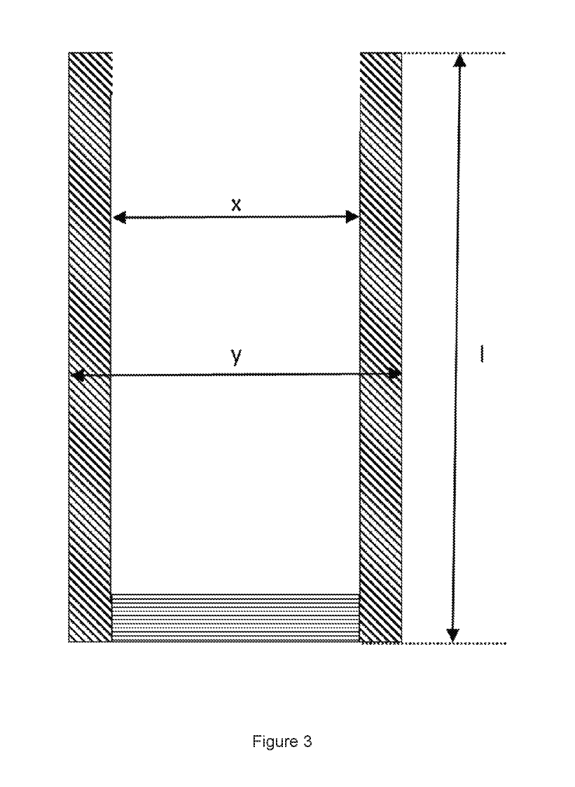

The term “inner wall cylinder diameter” is defined as the diameter of the cylinder, measured from inner wall to inner wall (“x” in FIG. 3), whereas the “outer wall cylinder diameter” is the diameter of the cylinder, measured from outer wall to outer wall (“y” in FIG. 3). In order for the collecting container (3) to be suitable for receiving the membrane adsorber unit (2) and in order for the membrane adsorber unit (2) to be suitable for receiving the prefilter unit (1), the inner wall cylinder diameter of the collecting container (3) is larger than the outer wall cylinder diameter of the membrane adsorber unit (2), while the inner wall cylinder diameter of the membrane adsorber unit (2) in turn is larger than the outer wall cylinder diameter of the prefilter unit (1). According to a preferred embodiment, the inner wall cylinder diameter of the collecting container (3) is slightly, i.e., no more than 1 mm, larger than the outer wall cylinder diameter of the membrane adsorber unit (2); and the inner wall cylinder diameter of the membrane adsorber unit (2) is slightly, i.e., no more than 1 mm, larger than the outer wall cylinder diameter of the prefilter unit (1). In this way it is ensured that, on the one hand, the components can be inserted with a precise fit and, as a result, do not impinge on each other during centrifugation and, in so doing, are thereby damaged; and, on the other hand, a maximum volume of protein-containing fluid can be received in the prefilter unit (1) prior to centrifugation.

The outer wall cylinder diameter of the collecting container (3) is not subject to any particular constraint. Preferably the outer wall cylinder diameter of the collecting container (3) of the device provided herein is chosen in such a way that the device can be used in a commercially available centrifuge.

The length (“l” in FIG. 3) of the cylindrically shaped prefilter unit (1), the cylindrically shaped membrane adsorber unit (2) and the cylindrically shaped collecting container (3) is not subject to any particular constraint. Preferably the overall length of the device provided herein in the “fitted-together state” in FIGS. 2A-2B is chosen in such a way that the device can be used in a commercially available centrifuge.

According to a preferred embodiment, the lengths of the prefilter unit (1) and the membrane adsorber unit (2) are about half as long as the length of the collecting container (3), with the length of the prefilter unit (1) being slightly shorter than the length of the membrane absorber unit (2). In this case in the “fitted-together state” of the device in FIGS. 2A-2B, the prefilter unit (1) can receive approximately the same volume of fluid as the region of the collecting container (3), which is not occupied by the prefilter unit (1) and the membrane absorber unit (2). In this way it is ensured that a maximum quantity of protein-containing fluid can be used in a centrifugation process without any fluid residue remaining in the prefilter unit (1) at the end of centrifugation. According to a particularly preferred embodiment of the present invention, the prefilter unit (1) and the collecting container (3) are configured in such a way that the collecting container (3) is suitable for receiving about 1 to about 50 ml of fluid (for example, about 10 to about 40 ml), and the prefilter unit (1) is suitable for receiving about 0.5 to about 25 ml of fluid. In this way it is ensured that during a centrifugation process of the device provided herein in the “fitted-together state” in FIGS. 2A-2B, a maximum quantity of protein-containing fluid can be used in a commercially available, standard centrifuge without any fluid residue remaining in the prefilter unit (1) at the end of centrifugation.

The cylindrically shaped prefilter unit (1) of the device provided herein is suitable for receiving the protein-containing fluid prior to centrifugation and comprises at least one size exclusion filter (4). It is advantageous to have at least one size exclusion filter (4) retain cell debris and other constituents of the protein-containing fluid that would either cake the porous membrane (5) and, in so doing, would render it useless for adsorbing the protein, or would contaminate the eluted protein solution during centrifugation of the device in the fitted-together state. As a result, it is possible to use a protein-containing fluid for purification in an advantageous way, wherein said protein-containing fluid is a cell broth that was not subjected to any further pre-purification/filtration.

Size exclusion filters (4) in the context of the present application are depth filters or microfilters and ultrafilters, as defined in Pure Appl. Chem., 1996, 68, 1479. In this case microfiltration is defined as a pressure-driven, membrane-based separation process, in which particles and detached macromolecules larger than 0.1 μm are retained. In contrast, ultrafiltration is defined as a pressure-driven, membrane-based separation process, in which particles and detached macromolecules smaller than 0.1 μm and larger than about 2 nm are retained. Suitable materials for size exclusion filters include any conventional membrane and filter material of either a polymeric nature or inorganic composition (for example, glass fiber), for example, polymers of cellulose, cellulose derivatives, such as cellulose acetate and cellulose nitrate, polypropylene, polyethylene, polycarbonate, polyether ketones, polyvinylidene fluoride, polysulfones, polyether sulfones, aromatic and aliphatic polyamides, ceramics, such as Al₂O₃, silicates, nitrides, borides and mixtures of these substances, i.e., materials, from which porous, mechanically integral shaped bodies can be produced. It is also possible to use conventional filter aids, such as, for example, diatomaceous earth, as a separation aid.

According to a preferred embodiment, the size exclusion limit (90% cut-off) of the size exclusion filter (4) of the device is greater than 0.2 μm, so that the particulate constituents cannot nest in the downstream porous membrane (5). According to a preferred embodiment, the size exclusion limit of the size exclusion filter (4) is in a range of 0.05 μm to 200 μm, more preferably 0.1 μm to 10 μm. The thickness of the size exclusion filter (4) is not subject to any particular constraint. According to a preferred embodiment, the thickness is in a range of 50 μm to 2 cm, preferably from 100 μm to 0.5 cm. According to one embodiment, the cylindrically shaped prefilter unit (1) comprises two or more size exclusion filters (4), which are arranged preferably adjacent. In this embodiment it is advantageous that a gradient is possible, for example, a coarse size exclusion filter (for example, a depth filter) followed by a finer size exclusion filter, an arrangement that leads advantageously to a high working capacity and a high throughput.

The at least one size exclusion filter (4) is arranged perpendicular to the longitudinal direction (“l” in FIG. 3) of the cylindrically shaped prefilter unit (1). The position of the size exclusion filter (4) in the longitudinal direction is not subject to any particular constraint. According to a preferred embodiment, the at least one size exclusion filter (4) is arranged at one end of the cylindrically shaped prefilter unit (1), in particular, at the end of the prefilter unit (1) that faces in the direction of the membrane adsorber unit (2) in the “fitted-together state” of the device, as shown in FIGS. 1 and 2A-2B, since in this way it is ensured that a maximum volume of protein-containing fluid can be received in the prefilter unit (1) prior to centrifugation.

According to a preferred embodiment, the prefilter unit may include three layers or three size exclusion filters. These filters may be arranged adjacent to each other. For example, in some aspects, the prefilter unit may comprise two glass fiber layers (an upper fiber glass layer and a middle glass fiber layer) and a lower asymmetric PES-membrane-layer. In this example, the pore sizes of the two glass fiber layers may range from about 1.5 μm to about 3.25 μm and from about 0.8 μm to about 1.4 μm, and the pore size of the asymmetric polyethersulfone (PES)-membrane-layer may range from about 0.15 μm to about 0.3 μm, or preferably, may be about 0.22 μm. In some aspects, the upper fiber glass layer may range from about 1.5 μm to about 3.25 μm, the middle fiber glass layer may range from about 0.8 μm to about 1.4 μm, and the lower asymmetric PES-membrane-layer may range from about 0.15 μm to about 0.3 μm, or preferably, may be about 0.22 μm. In this example, upper layer refers to the maximum distance from the porous membrane (5), lower layer refers to the minimum distance from porous membrane (5), and middle layer refers to a layer positioned between the upper layer and lower layer. In some cases, the PES membrane is preferably sealed to prevent the protein-containing fluid from passing unfiltered at the edge of the membrane. In some aspects, an asymmetric polyethersulfone (PES)-membrane-layer may have pores that are larger on one side of the membrane than on the other side of the membrane.

The cylindrically shaped membrane absorber unit (2) of the device comprises at least one porous membrane (5) with functional constituents, i.e., with chemical groups, which adsorb (preferably selectively) the protein that is to be purified. It is advantageous to have the porous membrane (5) bind the protein to be purified during convective flow. The functional constituents or, more specifically, the chemical groups may be present either originally in/on the porous membrane (5) or may be introduced subsequently into/on the porous membrane (5) by methods known to the person skilled in the art. In the context of the present application, the term “chemical groups” shall mean both the groups of atoms/ions/molecules and the individual atoms or ions that are able to adsorb the protein that is to be purified. Such functional constituents or rather chemical groups and also their introduction into/on a porous membrane are known to the skilled person and are employed as a function of the type of protein to be purified. Examples of functional constituents or, more specifically, chemical groups of the porous membrane (5) for adsorbing the protein to be purified may include Ni²⁺, Cu²⁺, protein A, streptavidin or derivatives, and amylose. According to a preferred embodiment, the chemical groups comprise Ni²⁺ and Cu²⁺ ions that are suitable for adsorbing His-tagged proteins.

The porous membrane (5) in the membrane adsorber unit (2) may be constructed of either a single layer or two or more membrane layers. The more membrane layers there are in the membrane adsorber unit (2), the higher the total binding capacity of the membrane adsorber unit (2) will be. However, if too many membrane layers are used, the elution volume becomes too high. Consequently it is advantageous to have the porous membrane (5) constructed of 3 to 30 membrane layers, more preferably 5 to 25 membrane layers. The skeletal material of the porous membrane (5) is not subject to any particular constraint; and it is possible to use, for example, membranes made of cellulose hydrate/ester/acetate, cellulose nanofibers, nylon nanofibers, polyethylene terephthalate (PET), poly(oxy-1,4-phenylsulfonyl-1,4-phenyl), polyamides, polyesters, polyvinylidene fluoride (PVDF), polyethylene (PE), polypropylene (PP) and/or hydrogel-modified membranes.

The thickness of the porous membrane (5) is not subject to any particular constraint. According to a preferred embodiment, the thickness is in a range of 50 μm to 2 cm, preferably 100 μm to 500 mμm. The pore size of the porous membrane (5) is not subject to any particular constraint. According to a preferred embodiment, the pore size is in a range of 0.1 μm to 10 μm. If the porous membrane (5) is constructed of two or more membrane layers, then these membrane layers may have different or the same membrane thicknesses and/or pore sizes.

According to further aspects, the porous membrane (5) is arranged perpendicular to the longitudinal direction (“l” in FIG. 3) of the cylindrically shaped membrane adsorber unit (2). The position of the porous membrane (5) in the longitudinal direction is not subject to any particular constraint. According to a preferred embodiment, the porous membrane (5) is arranged at one end of the cylindrically shaped membrane adsorber unit (2), in particular, at the end of the membrane adsorber unit (2) that faces in the direction of the collecting container (3) in the “fitted-together state” of the device, as shown in FIGS. 1 and 2A-B, since in this way it is ensured that the largest possible prefilter unit (1) and, thus, also the maximum possible volume of fluid present in the prefilter unit (1) can be received in the membrane adsorber unit (2).

It is advantageous that, in contrast to the chromatographic materials that are often used in the prior art, such as gels packed into columns, the porous membrane (5) can be stored in a dry place (i.e. not immersed in a liquid) and at room temperature without the porous membrane (5) losing its stability. Due to the fact that the porous membrane (5) can be sterilized with simple methods (for example, by irradiation with gamma radiation), bacterial contamination can be easily avoided in an advantageous way.

According to a preferred embodiment, the device also comprises a flow restrictor. The term “flow restrictor” is defined as a component that is configured in such a way that during centrifugation of the device the dwell time of the protein-containing fluid in the at least one porous membrane layer is increased. This feature leads advantageously to an enhanced adsorption of the protein to be purified on the porous membrane (5) of the cylindrically shaped membrane adsorber unit (2).

According to a particularly preferred embodiment, the flow restrictor is arranged in the cylindrically shaped membrane adsorber unit (2), in particular, adjacent to the porous membrane (5), in particular, on the side of the porous membrane (5) that faces in the direction of the collecting container (3) in the “fitted-together state” of the device. According to the most preferred embodiment, the flow restrictor is arranged at one end of the membrane adsorber unit (2); and the porous membrane (5) is arranged immediately adjacent to the flow restrictor in the direction of the center of the cylinder in the longitudinal direction of the cylindrically shaped membrane adsorber unit (2). Thus, on the one hand, it is ensured that the conditions for an enhanced adsorption of the protein to be purified on the porous membrane (5) have been met due to the longer dwell time of the protein-containing fluid; and, on the other hand, it is ensured that the largest possible prefilter unit (1) and, thus, also the maximum possible volume of fluid, present in the prefilter unit (1), can be received in the membrane adsorber unit (2).

The design of the flow restrictor is not subject to any particular constraint, as long as the flow restrictor lends itself to increasing the dwell time of the protein-containing fluid in the porous membrane (5) during centrifugation. Examples of the flow restrictor include membranes, filters, ultrafilters and microfilters (for example, 0.1 μm pore size having no more than 500 kDa, so that retention of the protein does not occur).

According to a further preferred embodiment, the flow restrictor is at least one membrane, in particular, a membrane, which exhibits less porosity than the at least one porous membrane (5) of the membrane adsorber unit (2), and, thus, reduces the flow rate of the fluid. Membrane materials that can be used in this context include, for example, the materials listed above with respect to the porous membrane (5). The thickness of the membrane is not subject to any particular constraint. According to a preferred embodiment, the thickness is in a range of 50 μm to 3 cm, preferably 100 μm to 2 cm. The pore size of the membrane is not subject to any particular constraint. According to a preferred embodiment, the pore size is in a range of 0.05 μm up to the pore size of the porous membrane (5). If the flow restrictor comprises two or more membranes, then these membranes may have different or the same membrane thicknesses and/or pore sizes.

The cylindrically shaped collecting container (3) of the device is not subject to any particular constraint, as long as the collecting container (3) is closed at one end and thus can collect the filtrate. The closed end of the cylindrically shaped collecting container (3) may taper, for example, to a truncated cone, as shown in FIGS. 1 and 2A-2B.

According to one embodiment, the device comprises an end cap (6), with which the prefilter unit (1) and/or the membrane adsorber unit (2) can be reversibly closed. In this way it is ensured that during centrifugation of the device in the “fitted-together state” no fluid escapes from the device. The end cap (6) can be either a separate component or can be attached to the prefilter unit (1), the membrane adsorber unit (2) or the collecting container (3) in a manner allowing movement, for example, via film hinges. According to a preferred embodiment, the end cap (6) is secured to the end of the membrane adsorber unit (2), as shown in FIGS. 1 and 2A-2B, said end being opposite the porous membrane (5)/optional flow restrictor.

An additional aspect relates to a method for purifying protein from a protein-containing fluid, said method comprising the steps of:
(a) providing a device for purifying protein from a protein-containing fluid, wherein a prefilter unit (1) is located inside or partially inside a membrane adsorber unit (2), and the membrane adsorber unit (2) is located inside or partially inside a collecting container (3);
(b) filling the prefilter unit (1) with the protein-containing fluid;
(c) centrifuging the device, wherein constituents of the protein-containing fluid that cake a porous membrane (5) of the membrane absorber (2) are retained by at least one size exclusion filter (4) of the prefilter unit (1); the protein to be purified is adsorbed on the porous membrane (5) of the membrane adsorber unit (2); and a filtrate collects in the collecting container (3);
(d) discarding the filtrate located in the collecting container (3);
(f) filling the membrane adsorber unit (2) with elution buffer solution;
(g) eluting the protein, adsorbed on the porous membrane (5), with the elution buffer solution in the collecting container (3), to obtain purified protein.

In step (a) of the method, the device, described above, is provided, wherein the device is in the “fitted-together state”, as shown in FIGS. 2A-2B. In the fitted together state, the prefilter unit (1) is located inside or partially inside the membrane adsorber unit (2); and the membrane adsorber unit (2) is located inside or partially inside the collecting container (3).

In step (b) of the method, the prefilter unit (1) is filled with protein-containing fluid. It is advantageous to be able to use as the protein-containing fluid a cell broth (either with lysed cells or with intact cells), which had not been subjected to further purification/filtration as customary in the prior art and, therefore, contains cell debris and other constituents, which could possibly cake the porous membrane (5) (or even other standard chromatographic materials). This is possible, because the device in its modular design also comprises at least one size exclusion filter (4), which protects the porous membrane (5) from being caked, in the prefilter unit (1).

In step (c) of the method, the device, which is filled with a protein-containing fluid, is centrifuged. In this case the constituents of the protein-containing fluid that could potentially cake the porous membrane (5) are retained by the at least one size exclusion filter (4), while the protein to be purified passes through the size exclusion filter (4) unimpeded. The protein to be purified is adsorbed (selectively) onto the porous membrane (5) of the membrane adsorber unit (2), with said membrane following the size exclusion filter (4), while the filtrate, separated from the protein, collects in the collecting container (3).

The centrifugation time and speed in step (c) is not subject to any particular constraint and can be readily chosen by a skilled person in such a way that the fluid, which may be found in the prefilter unit (1) at the start of centrifugation, is moved completely through the size exclusion filter (4) and the porous membrane (5) and collects as the filtrate in the collecting container (3). In addition, it is known to the skilled person that the centrifugation speed should not be too high, since in this case the complete adsorption of the protein to be purified on the porous membrane (5) cannot be guaranteed.

In step (d) of the method, the prefilter unit (1) and the filtrate, located in the collecting container (3), are discarded. The term “discarded” shall mean that the prefilter unit (1) is either disposed or recovered (cleaned) again, for reuse with an additional purification process. The filtrate is also either disposed or can be fed optionally to a re-purification process, in order to isolate any small amounts of residue of the protein to be purified that may still be present in the filtrate. The re-purification may be carried out, for example, immediately once again by using the same membrane (5); or a new device can be used for this purpose.

After step (d) the method comprises optionally step (e). In the optional step (e) the membrane adsorber unit (2) is filled with a wash buffer solution; the device is centrifuged without the discarded prefilter unit (1), so that the wash buffer solution collects in the collecting container (3). Then the wash buffer solution, which is located in the collecting container (3), is discarded. The optional step (e) may be carried once or also multiple times and removes in an advantageous way the constituents of the original protein-containing fluid that are either unbound or non-specifically bound to the porous membrane (5). Wash buffer solutions are known to the person skilled in the art; and it is possible to use, for example, buffers having the desired pH value and the desired salt concentration as the wash buffer solution.

In step (f), wherein said step (f) follows either step (d) or the optional step (e), the membrane adsorber unit is filled with the elution buffer solution. The skilled person may select, as a function of the protein to be purified and the porous membrane (5) that is to be used, an elution buffer solution that is able to elute the adsorbed protein from the porous membrane (5), for example, a wash buffer having a competitive molecule (for example, imidazole, biotin or maltose), which can dislodge the protein from the porous membrane (5). As an alternative, an elution buffer with a low pH value can also be used.

In step (g) of the method, the protein, which is adsorbed on the porous membrane (5), is eluted with the elution buffer solution in the collecting container (3). In this case the purified protein is obtained as the filtrate in the collecting container (3). Preferably the elution takes place under centrifugation.

Since the method achieves the necessary flow rate to purify the protein by centrifugation, there is no need to apply negative pressure or rather a vacuum. Consequently it is advantageous that all of the steps of the method provided herein can be carried out under standard atmospheric pressure.

Another aspect relates to the application of the device for purifying protein from a protein-containing fluid. According to a preferred embodiment, the protein to be purified is a His-tagged (His-marked) protein or an antibody (for example, mbp-tagged or strep-tagged).

The devices and methods provided herein make it possible to carry out in an advantageous way both a size exclusion filtration and a protein adsorption simultaneously in just one centrifugation step, in order to purify the target protein. In this way it is possible to purify protein from an unclarified cell broth, so that advantageously no pre-filtration steps are necessary. Since the necessary flow rate for purifying the protein by centrifugation is achieved with the device provided herein, it is not necessary to apply negative pressure or rather a vacuum, so that the protein-containing fluid is protected against damage. In addition, this arrangement simplifies the design of the protein purification device provided herein, since the device does not have to have a connecting system, in order to generate negative pressure. Owing to the compact, modular design of the device with a membrane adsorber, instead of a chromatography column, a significantly larger volume of protein-containing fluid per centrifugation operation can be purified than with the devices, which are known from the prior art and which are suitable for centrifugation. In addition, it is advantageous that the porous membrane (5) of the device can be stored in a dry place and at room temperature without any loss of stability. In addition, it is easy to avoid bacterial contamination due to the ease with which the device can be sterilized. Thus, the present devices and methods allow protein from a protein-containing fluid to be purified quickly, effectively, reliably and aseptically in an advantageous way.

### LIST OF REFERENCE NUMERALS

1 cylindrically shaped prefilter unit

2 cylindrically shaped membrane absorber unit

3 cylindrically shaped collecting container

4 size exclusion filter

5 porous membrane

6 end cap

What is claimed is:

1. A device for purifying protein from a protein-containing fluid, comprising:
a cylindrically shaped prefilter unit, which has at least one size exclusion filter;
a cylindrically shaped membrane adsorber unit, which has at least one porous membrane with functional constituents that adsorb the protein to be purified; and
a cylindrically shaped collecting container;
wherein the collecting container is suitable for receiving the membrane adsorber unit and the membrane adsorber unit is suitable for receiving the prefilter unit.

2. The device of claim 1, wherein the prefilter unit is suitable for receiving about 0.5 to about 25 ml of the fluid, and the collecting container is suitable for receiving about 1 to about 50 ml of the fluid.

3. The device of claim 1, wherein the prefilter comprises three filters, including an upper glass fiber filter having a pore size ranging from about 1.5 μm to about 3.25 μm, a middle glass fiber filter having a pore size ranging from about 0.8 μm to about 1.4 μm, and a lower asymmetric PES-membrane filter having a pore size of about 0.22 μm.

4. The device of claim 1, wherein the size exclusion filter is arranged at one end of the cylindrically shaped prefilter unit.

5. The device of claim 1, wherein the functional constituents of the porous membrane of the cylindrically shaped membrane adsorber unit are selected from Ni²⁺, Cu²⁺, protein A, streptavidin, streptavidin derivatives, or amylose.

6. The device of claim 1, wherein the porous membrane of the cylindrically shaped membrane adsorber unit is constructed of two or more membrane layers.

7. The device of claim 1, wherein the porous membrane is arranged at one end of the cylindrically shaped membrane adsorber unit.

8. The device of claim 1, further comprising a flow restrictor that is configured to increase a dwell time of the protein-containing fluid in the porous membrane during centrifugation of the device.

9. The device of claim 8, wherein the flow restrictor is arranged adjacent to the porous membrane of the membrane adsorber unit.

10. The device of claim 8, wherein the flow restrictor comprises at least one membrane that exhibits less porosity than the porous membrane of the membrane adsorber unit.

11. A method for purifying protein from a protein-containing fluid comprising the steps of:
providing a device comprising a prefilter unit containing at least one size exclusion filter, a membrane adsorber unit containing a porous membrane, and a collecting container, wherein the prefilter unit is located inside or partially inside the membrane adsorber unit, and the membrane adsorber unit is located inside or partially inside the collecting container;
filling the prefilter unit with the protein-containing fluid;
centrifuging the device, wherein constituents of the protein-containing fluid that cake the porous membrane of the membrane adsorber unit are retained by the at least one size exclusion filter, and the protein is adsorbed on the porous membrane of the membrane adsorber unit and a filtrate collects in the collecting container;
removing the prefilter unit and the filtrate located in the collecting container;
filling the membrane adsorber unit with elution buffer solution;
eluting the protein adsorbed on the porous membrane with the elution buffer solution in the collecting container, to obtain purified protein.

12. The method of claim 11, further comprising, after removing the prefilter unit and the filtrate and prior to filling the membrane adsorber unit with elution buffer solution, the additional steps of:
filling the membrane adsorber unit with a wash buffer solution;
centrifuging the device without the prefilter unit, such that the wash buffer solution collects in the collecting container, and then discarding the wash buffer solution located in the collecting container.

13. The method of claim 12, wherein eluting the protein takes place under centrifugation.

14. The method of claim 12, wherein all of the steps are carried out under standard atmospheric pressure.

15. The method of claim 12, wherein the protein is from a protein-containing fluid produced by cells.

16. The method of claim 12, wherein the protein to be purified is a His-tagged protein or a labeled antibody.
